(19) United States
(12) Patent Application Publication
(10) Pub. No.: US 2005/0061331 A1
(43) Pub. Date: Mar. 24, 2005

(54) Sanitary arm sleeve structure

(76) Inventor: Ken Cheung, Chino Hills, CA (US)

Correspondence Address: Raymond Y. Chan, Suite 128, 108 N. Ynez Ave., Monterey Park, CA, 91754, US

(21) Appl. No.: 10/667,038

(22) Filed: Sep. 22, 2003

(51) Int. Cl. A61B 19/00

(52) U.S. Cl. 128/849; 128/856

(57) ABSTRACT

A sanitary arm sleeve structure includes an arm sleeve, adapted for receiving the user's arm therewithin, having an outer side, an opposed inner side, and a free edge end, and a sleeve protection layer having an upper edge end attached on the arm sleeve and a lower edge end extended to the free edge end of the arm sleeve so as to overlappedly encircle the sleeve protection layer around the outer side of the arm sleeve. An elastic retaining loop is provided at the inner side of the arm sleeve above the free edge end thereof for applying an elastic binding force around the user's arm above the wrist thereof, so as to retain the arm sleeve in position that the sleeve protection layer is capable of encircling around the user's arm.

### BACKGROUND OF THE PRESENT INVENTION

1. Field of Invention

The present invention relates to an arm sleeve, and more particularly to a sanitary arm sleeve structure for using in the clinical profession, which provides a cleanable protective layer at a lower arm portion of the arm sleeve so as to prevent bacterial and dirt coated on the arm sleeve.

2. Description of Related Arts

During a physical check up or surgeon, doctors or nurses require contacting with patient such that they must wear a surgeon glove and lab coat for preventing body contamination. The surgeon glove, which is made of plastic, is disposable such that the physician is able to throw away the surgeon glove after every medical operation to prevent hand contamination. However, body fluids, blood, or human excrements may accidentally coated at the sleeve of the lab coat, especially during surgeon. It is wasteful that the lab coat is made of disposable material to prevent the bacterial and dirt coated on the sleeve.

Generally, surgeon doctors must wash the lab coat every after surgeon operation for hygiene purpose. However, body fluids or blood from the patient may spilt into the sleeve so as to contact with the arm of the doctor during surgeon. Therefore, the surgeon is unsanitary because the skin of the surgeon doctor often coated with contaminates. The clinic doctor also has the above problem that the body fluids or blood from the patient may contact with the arm of the doctor through the opening of the sleeve during diagnosis. Since hundreds of patients may visit the clinic doctor everyday, the contaminates coated at the sleeve and/or the arm may easily pass from the previous patient to the following patient.

For hygiene purpose, the doctor may wear a sleeve protection at the lower arm portion in order to protect any infection from the patient. Accordingly, the sleeve protection, which is made of washable material, has two elastic ends wherein when the arm of the user insert into the sleeve protection, the two elastic ends of the sleeve protection bind around the user's arm below the elbow and above the wrist respectively so as to retain the sleeve protection at the lower arm of the user. Such conventional sleeve protection can prevent the contaminates coating on the sleeve. However, the sleeve protection has several drawbacks.

Since the two elastic ends of the sleeve protection must be securely bound at the elbow and the wrist of the arm respectively, the movement of the arm is restricted by the elastic ends of the sleeve protection. However, the doctor requires a precise arm movement during surgeon or diagnosis. In addition, when the doctor stretch his or her arm, the elastic ends of the sleeve protection may force to slide along the arm, such that the doctor must adjust the position of each of the elastic ends of the sleeve protection. Thus, it is a hassle to wear the sleeve protection because the doctor must put the sleeve protection at the outer side of the sleeve of the lab coat, which will destroy the professional appearance of the doctor. Therefore, the sleeve protection is disadvantage in practice use.

### SUMMARY OF THE PRESENT INVENTION

A main object of the present invention is to provide a sanitary arm sleeve structure for using in the clinical profession, which provides a cleanable protective layer at a lower arm portion of the arm sleeve so as to prevent bacterial and dirt coated on the arm sleeve.

Another object of the present invention is to provide a sanitary arm sleeve structure which comprises a sleeve protection layer integrally and overlappedly formed at the outer side of the arm sleeve, so as to prevent the contaminates coating on the arm sleeve.

Another object of the present invention is to provide a sanitary arm sleeve structure which comprises an elastic retaining loop for applying an elastic binding force around the arm of the user so as to retain the sleeve protection layer in position with respect to the user's arm. In other words, the sleeve protection layer will retain at its position even the user stretch his or her arm.

Another object of the present invention is to provide a sanitary arm sleeve structure, wherein the retaining loop is positioned at an inner side of the arm sleeve to bind above the wrist of the user. Therefore, the retaining loop is hidden inside the arm sleeve to keep the professional appearance of the arm sleeve.

Another object of the present invention is to provide a sanitary arm sleeve structure, which does not require altering the original structure of the arm sleeve so as to minimize the manufacturing cost of the arm sleeve.

Another object of the present invention is to provide a sanitary arm sleeve structure, which is easy to use by simply inserting the user's arm into the arm sleeve.

Accordingly, in order to accomplish the above objects, the present invention provides a sanitary arm sleeve structure for a user's arm inserting thereinto, comprising:
an arm sleeve, adapted for receiving the user's arm therewithin, having an outer side, an opposed inner side, and a free edge end;
a sleeve protection layer having an upper edge end attached on the arm sleeve and a lower edge end extended to the free edge end of the arm sleeve so as to overlappedly encircle the sleeve protection layer around the outer side of the arm sleeve; and
an elastic retaining loop formed at the inner side of the arm sleeve above the free edge end thereof for applying an elastic binding force around the user's arm above the wrist thereof, so as to retain the arm sleeve in position that the sleeve protection layer is capable of encircling around the user's arm.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of a sanitary arm sleeve structure according to a preferred embodiment of the present invention, illustrating the sanitary arm sleeve structure designed for lab coat.

FIG. 2 is a sectional view of the sanitary arm sleeve structure according to the above preferred embodiment of the present invention.

FIG. 3 illustrates an alternative mode of the sanitary arm sleeve structure according to the above preferred embodiment of the present invention.

FIG. 4 illustrates the sanitary arm sleeve structure utilized with a surgeon coat according to the above preferred embodiment of the present invention.

### DETAILED DESCRIPTION OF THE PREFERRED EMBODIMENT

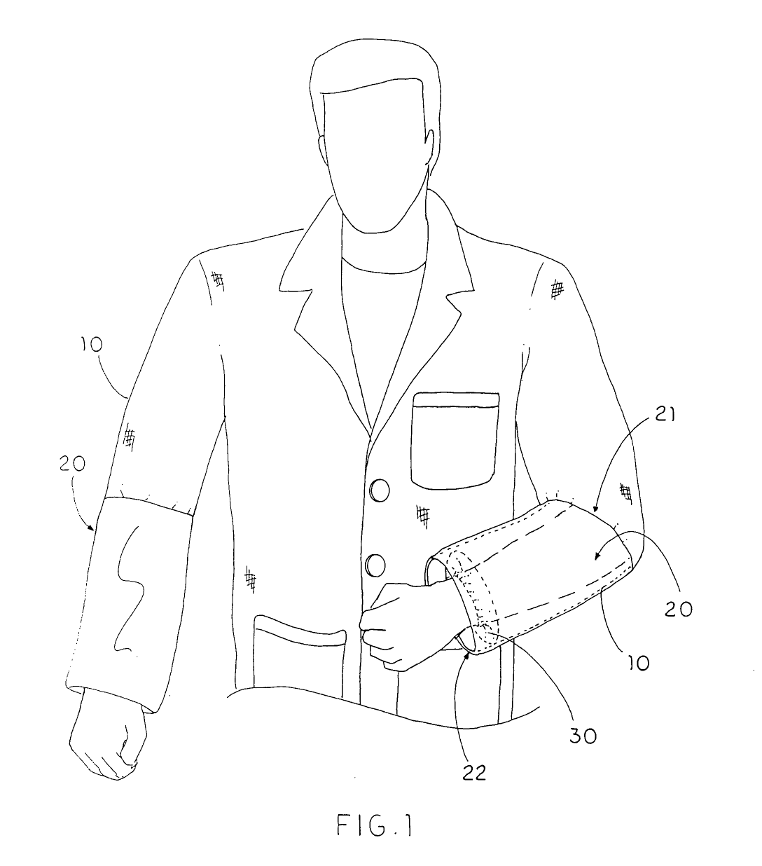

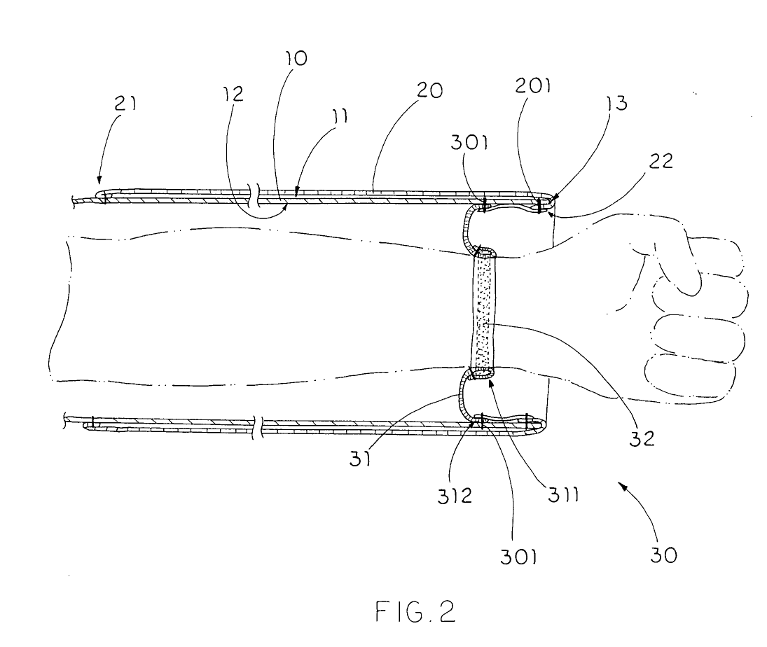

Referring to FIGS. 1 and 2 of the drawings, a sanitary arm sleeve structure utilized with a lab coat according to a preferred embodiment of the present invention is illustrated, wherein the sanitary arm sleeve structure comprises an arm sleeve 10, adapted for receiving the user's arm therewithin, having an outer side 11, an opposed inner side 12, and a free edge end 13.

The sanitary arm sleeve structure further comprises a sleeve protection layer 20 having an upper edge end 21 attached on the arm sleeve 10 and a lower edge end 22 extended to the free edge end 13 of the arm sleeve 10 so as to overlappedly encircle the sleeve protection layer 20 around the outer side 11 of the arm sleeve 10, and an elastic retaining loop 30 formed at the inner side 12 of the arm sleeve 10 above the free edge end 13 thereof for applying an elastic binding force around the user's arm above the wrist thereof, so as to retain the arm sleeve 10 in position that the sleeve protection layer 20 is capable of encircling around the user's arm.

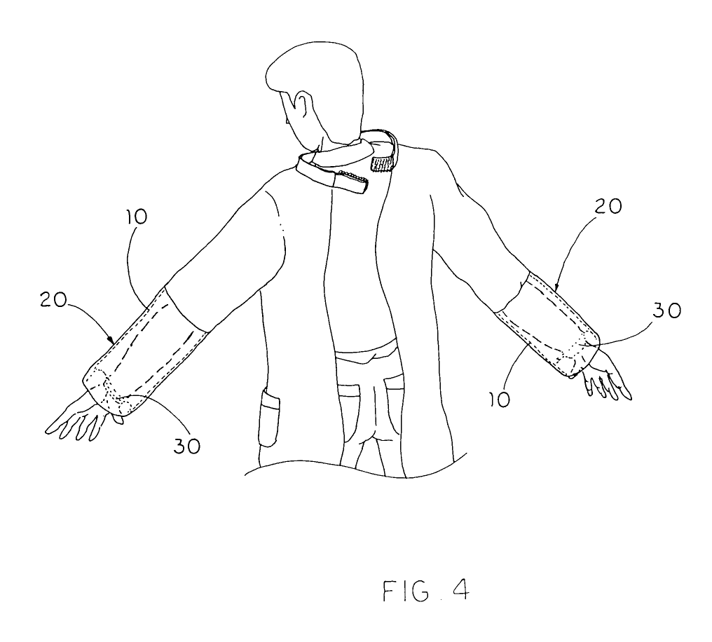

According to the preferred embodiment, when the user, such as a physician, wear the lab coat, the arm of the user inserts into the arm sleeve 10 that the free edge end 13 of the arm sleeve 10 is positioned below the wrist of the user's arm such that the arm sleeve 10 is arranged to encircle around the user's arm to prevent body contamination. Alternatively, the sanitary arm sleeve structure can be utilized with a surgeon coat, as shown in FIG. 4. The arm sleeve 10, which is made of fabric material such as cotton, is embodied as a part of the lab coat that the arm sleeve 10 is extended from a coat body.

The sleeve protection layer 20 has a tubular shape overlappedly cover on the outer side 11 of the arm sleeve 10 at a lower portion thereof wherein the upper edge end 21 of the sleeve protection layer 20 is securely attached on the arm sleeve 10 at a position below an elbow of the user's arm. The sleeve protection layer 20 is preferably made of silicone rubber having anti-adhesive properties, low chemical reactivity and low toxicity. In addition, the silicone rubber made sleeve protective layer 20 can be cleanable which is capable of substantially preventing the contaminates coating thereon.

Accordingly, the upper edge end 21 of the sleeve protection layer 20 is attached to the outer side 11 of the arm sleeve 10 by ultrasonic welding or sewing to substantially overlap the sleeve protection layer 20 on the outer side 11 of the arm sleeve 10.

As shown in FIG. 2, the lower edge end 22 of the sleeve protection layer 20 is inwardly folded to overlap on the inner side 12 of the arm sleeve 10 along the free edge end 13 thereof, wherein at least one line of attaching spots 201 is continuously aligned along the free edge end 13 of the arm sleeve 10 at the inner side 12 thereof to securely attach the lower edge end 22 of the sleeve protection layer 20 to the inner side 12 of the arm sleeve 10 such that the lower edge end 22 of the sleeve protection layer 20 is protectively encircling around the free edge end 13 of the arm sleeve 10. Accordingly, the line of attaching spots 201 can be embodied as a line of sewing spots or a line of ultrasonic welding spots.

The retaining loop 30 comprises a blocking strap 31 having an inner edge 311 and an outer edge 312 extended from the inner side 12 of the arm sleeve 10 at an edge portion 14 thereof, and a circular elastic element 32 provided around the inner edge 311 of the blocking strap 31 for providing the elastic binding force at the user's arm, wherein a diameter of the inner edge 311 of the blocking strap 31 is smaller than that of the arm sleeve 10, such that when the elastic element 31 binds around the user's arm, the blocking strap 31 functions as a barrier for preventing an unwanted material, such as body fluid or blood, entering into the arm sleeve 10.

The blocking strap 31, which is also made of silicone rubber, has a circular shape defining the inner edge 311 forming as a pocket to receive the elastic element 32 therearound. The retaining loop 30 further comprises at least one line of affixing spots 301 continuously aligned along a length of the outer edge 312 of the blocking strap 31 so as to securely attach the blocking strap 31 on the inner side 12 of the arm sleeve 10. Accordingly, the line of affixing spots 301 can be embodied as a line of sewing spots or a line of ultrasonic welding spots.

The elastic element 32, according to the preferred embodiment, is an elastic band securely provided around the inner edge 311 of the blocking strap 31 for binding around the user's arm at a position above the wrist thereof. As shown in FIG. 2, a width of the edge portion 14 of the arm sleeve 10 is longer than a width of the blocking strap 31 such that the elastic element 32 is positioned within the arm sleeve 10 so as to keep the professional appearance of the lab coat. It is worth to mention that even the user stretch his or her arm, the elastic element 32 will remain inside the arm sleeve 10 so as to prevent the retaining loop 30 accidentally sliding out of the arm sleeve 10.

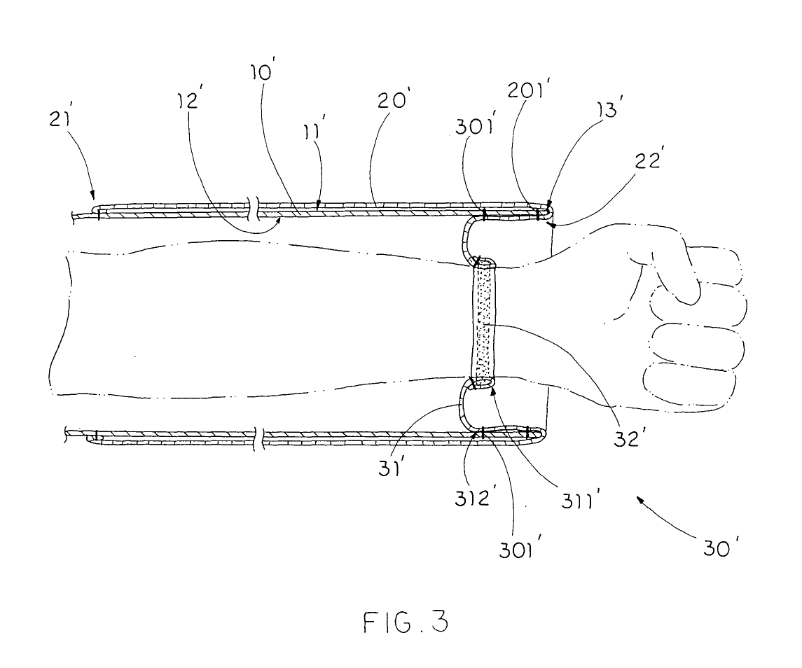

FIG. 3 illustrates an alternative mode of the sanitary arm sleeve structure wherein the arm sleeve 10′ has an outer side 11′, an opposed inner side 12′, and a free edge end 13′.

The sleeve protection layer 20′ has an upper edge end 21′ attached on the arm sleeve 10′ and a lower edge end 22′ extended to the free edge end 13′ of the arm sleeve 10′ so as to overlappedly encircle the sleeve protection layer 20′ around the outer side 11′ of the arm sleeve 10′.

The upper edge end 21′ of the sleeve protection layer 20′ is attached to the outer side 11′ of the arm sleeve 10′ by ultrasonic welding or sewing to substantially overlap the sleeve protection layer 20′ on the outer side 11′ of the arm sleeve 10′. The lower edge end 22′ of the sleeve protection layer 20′ is inwardly folded to overlap on the inner side 12′ of the arm sleeve 10′ along the free edge end 13′ thereof.

The retaining loop 30′ comprises a blocking strap 31′ having an inner edge 311′ and an outer edge 312′ extended from the inner side 12′ of the arm sleeve 10′ at an edge portion 14′ thereof, and a circular elastic element 32′ provided around the inner edge 311′ of the blocking strap 31′ for providing the elastic binding force at the user's arm.

As shown in FIG. 3, the outer edge 312′ of the blocking strap 31′ is integrally extended from the lower edge end 22′ of the sleeve protection layer 20′, wherein at least one line of affixing spots 301′ continuously aligned along a length of the outer edge 312′ of the blocking strap 31′ so as to securely attach the blocking strap 31′ on the inner side 12′ of the arm sleeve 10′. A width of the edge portion 14′ of the arm sleeve 10′ is longer than a width of the blocking strap 31′ such that the elastic element 32′ will remain inside the arm sleeve 10′ so as to prevent the retaining loop 30′ accidentally sliding out of the arm sleeve 10′.

It is worth to mention that the line of affixing spots 301′ not only securely affixes the blocking strap 31′ on the inner side 12′ of the arm sleeve 10′ but also firmly attaches the sleeve protection layer 20′ on the outer side 11′ of the arm sleeve 10′. In order to further securely retain the sleeve protection layer 20′ in position, at least one line of attaching sports 201′ is continuously aligned along the free edge end 13′ of the arm sleeve 10′ at the inner side 12′ thereof so as to securely overlap the sleeve protection layer 20′ on the outer side 11′ of the arm sleeve 10′.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure form such principles. Therefore, this invention includes all modifications encompassed within the spirit and scope of the following claims.

1. A sanitary arm sleeve structure for an arm of a user inserting thereinto, comprising:
an arm sleeve, adapted for receiving said user's arm therewithin, having an outer side, an opposed inner side, and a free edge end;
a sleeve protection layer having an upper edge end attached on said arm sleeve and a lower edge end extended to said free edge end of said arm sleeve so as to overlappedly encircle said sleeve protection layer around said outer side of said arm sleeve; and
an elastic retaining loop provided at said inner side of said arm sleeve above said free edge end thereof for applying an elastic binding force around said user's arm above a wrist thereof, so as to retain said arm sleeve in position that said sleeve protection layer is capable of encircling around said user's arm.

2. The sanitary arm sleeve structure, as recited in claim 1, wherein said retaining loop comprises a blocking strap having an inner edge and an outer edge extended from said inner side of said arm sleeve at an edge portion thereof, and a circular elastic element provided around said inner edge of said blocking strap for providing said elastic binding force at said user's arm, wherein a diameter of said inner edge of said blocking strap is smaller than that of said arm sleeve.

3. The sanitary arm sleeve structure, as recited in claim 2, wherein a width of said edge portion of said arm sleeve is longer than a width of said blocking strap so as to retain said elastic element within said arm sleeve at a position above said free edge end thereof.

4. The sanitary arm sleeve structure, as recited in claim 2, wherein said retaining loop further comprises at least one line of affixing spots continuously aligned along a length of said outer edge of said blocking strap so as to securely attach said blocking strap on said inner side of said arm sleeve.

5. The sanitary arm sleeve structure, as recited in claim 3, wherein said retaining loop further comprises at least one line of affixing spots continuously aligned along a length of said outer edge of said blocking strap so as to securely attach said blocking strap on said inner side of said arm sleeve.

6. The sanitary arm sleeve structure, as recited in claim 1, wherein said lower edge end of said sleeve protection layer is inwardly folded to overlap on said inner side of said arm sleeve along said free edge end thereof such that said lower edge end of said sleeve protection layer is protectively encircling around said free edge end of said arm sleeve.

7. The sanitary arm sleeve structure, as recited in claim 2, wherein said lower edge end of said sleeve protection layer is inwardly folded to overlap on said inner side of said arm sleeve along said free edge end thereof such that said lower edge end of said sleeve protection layer is protectively encircling around said free edge end of said arm sleeve.

8. The sanitary arm sleeve structure, as recited in claim 3, wherein said lower edge end of said sleeve protection layer is inwardly folded to overlap on said inner side of said arm sleeve along said free edge end thereof such that said lower edge end of said sleeve protection layer is protectively encircling around said free edge end of said arm sleeve.

9. The sanitary arm sleeve structure, as recited in claim 5, wherein said lower edge end of said sleeve protection layer is inwardly folded to overlap on said inner side of said arm sleeve along said free edge end thereof such that said lower edge end of said sleeve protection layer is protectively encircling around said free edge end of said arm sleeve.

10. The sanitary arm sleeve structure, as recited in claim 7, wherein said outer edge of said blocking strap is integrally extended from said lower edge end of said sleeve protection layer.

11. The sanitary arm sleeve structure, as recited in claim 8, wherein said outer edge of said blocking strap is integrally extended from said lower edge end of said sleeve protection layer.

12. The sanitary arm sleeve structure, as recited in claim 9, wherein said outer edge of said blocking strap is integrally extended from said lower edge end of said sleeve protection layer.

13. The sanitary arm sleeve structure, as recited in claim 6, wherein at least one line of attaching spots is continuously aligned along said free edge end of said arm sleeve at said inner side thereof to securely attach said lower edge end of said sleeve protection layer to said inner side of said arm sleeve.

14. The sanitary arm sleeve structure, as recited in claim 9, wherein at least one line of attaching spots is continuously aligned along said free edge end of said arm sleeve at said inner side thereof to securely attach said lower edge end of said sleeve protection layer to said inner side of said arm sleeve.

15. The sanitary arm sleeve structure, as recited in claim 10, wherein at least one line of attaching spots is continuously aligned along said free edge end of said arm sleeve at said inner side thereof to securely attach said lower edge end of said sleeve protection layer to said inner side of said arm sleeve.

16. The sanitary arm sleeve structure, as recited in claim 12, wherein at least one line of attaching spots is continuously aligned along said free edge end of said arm sleeve at said inner side thereof to securely attach said lower edge end of said sleeve protection layer to said inner side of said arm sleeve.

17. The sanitary arm sleeve structure, as recited in claim 1, wherein said sleeve protection layer is made of silicone rubber.

18. The sanitary arm sleeve structure, as recited in claim 6, wherein said sleeve protection layer is made of silicone rubber.

19. The sanitary arm sleeve structure, as recited in claim 9, wherein said sleeve protection layer is made of silicone rubber.

20. The sanitary arm sleeve structure, as recited in claim 16, wherein said sleeve protection layer is made of silicone rubber.
